(19) United States
(12) Patent Application Publication
(10) Pub. No.: US 2026/0265296 A1
(43) Pub. Date: Sep. 10, 2026

(54) METHODS FOR ENHANCING PROTEIN CRYSTALLIZATION BY BULK AMINO ACID SUBSTITUTION

(71) Applicants: John F. Hunt, New York, NY (US); Nooriel E. Banayan, Great Neck, NY (US)

(72) Inventors: John F. Hunt, New York, NY (US); Nooriel E. Banayan, Great Neck, NY (US); Blaine Loughlin, New York, NY (US); Shikha Singh, Faridabad (IN)

(21) Appl. No.: 19/166,308

(22) PCT Filed: Apr. 1, 2024
(86) PCT No.: PCT/US2024/022553

§ 371 Date: Sep. 17, 2025

Related U.S. Application Data
(60) Provisional application No. 63/493,300, filed on Mar. 30, 2023.

(51) Int. Cl. C07K 1/30

(52) U.S. Cl. CPC C07K 1/306

(57) ABSTRACT

Disclosed herein are certain amino acid substitutions shown to improve protein crystallization, including bulk lysine-to-arginine (KR), aspartate-to-glutamate (DE), and asparagine-to-glutamine (NQ) substitutions. The method ranks lysine, aspartate or glutamine sites in a target protein based on the redundancy-corrected KR, DE or NQ substitution frequencies in homologs. We found that three unrelated single-domain proteins can tolerate 5-11 KR substitutions with at most minor destabilization. For two of these three proteins, the construct with the largest number of KR substitutions exhibits significantly enhanced crystallization propensity. This approach rapidly produced a 1.9 Å crystal structure of a human protein domain refractory to crystallization with its native sequence. Structures from bulk-KR-substituted domains show the engineered arginine residues frequently make hydrogen-bonds across crystal-packing interfaces. Thus, bulk KR, DE, or NQ substitution represents a rational and efficient method for probabilistic engineering of protein surface properties to improve protein crystallization.

### STATEMENT REGARDING FEDERALLY SPONSORED RESEARCH

This invention was made with government support under GM127883 awarded by US NIH-NIGMS. The government has certain rights in the invention.

### FIELD OF THE INVENTION

This disclosure relates to methods and systems for evaluating and enhancing protein crystallization efficiency through bulk amino acid substitution including, but not limited to, lysine-to-arginine, aspartate-to-glutamate, and asparagine-to-glutamine substitution. The disclosed methods and systems enable the evaluation of amino acid sequences to predict the likelihood of specific amino acid substitutions enhancing protein crystallization propensity. This capability facilitates higher resolution crystallography and enables a more accurate determination of protein molecular structure.

### BACKGROUND

More than 50 years after the solution of the first protein crystal structure, protein crystallization remains a hit-or-miss proposition. Synergistic developments in crystallographic methods, synchrotron beamlines, and high-speed computing have made structure solution and refinement routine, even for massive complexes, but only if high-quality crystals are available. However, there has been comparatively little progress in improving methods for protein crystallization.

Structural genomics consortia systematically confirmed that most naturally occurring proteins do not readily yield high-quality crystals suitable for x-ray structure determination and that crystallization is the major obstacle to the determination of protein structures using diffraction methods. While numerous methods have been developed that have some efficacy in improving protein crystallization properties, none work with sufficiently high efficiency to have been applied with significant frequency by practicing crystallographers. The development of AlphaFold[1] has provided a de facto solution to the protein-folding problem for many sequence families; however, its occasional failure, limited stereochemical accuracy, and inability to date to model ligand complexes means protein crystallography remains widely practiced and frequently challenging, especially for structure-based drug discovery projects. Therefore, there has remained a need for more efficient methods for rational engineering of protein surface properties to improve crystallization propensity, which the present disclosure addresses herein.

### SUMMARY OF THE INVENTION

This invention is generally directed to methods for increasing the propensity of a naturally occurring protein to crystallize based on probabilistic engineering of protein surface properties while preserving protein stability, solubility, and conformation. The mutant proteins so obtained frequently yield high resolution crystallographic data and enable accurate determination of protein molecular structure.

Accordingly, a method is provided to improve the number and quality of obtainable protein crystal forms by mutationally substituting, on a naturally occurring protein refractive to crystallization, multiple native lysine residues with arginine residues (KR substitution); multiple native asparagine residues with glutamine residues (NQ substitution); or multiple native aspartate residues with glutamate residues (DE substitution). Sites for substitution may be selected based on the frequencies of the same substitutions being observed in naturally evolved proteins, with more frequently substituted sites being selected before less substituted sites. Alternatively, sites for substitution may be selected based on the redundancy-corrected frequencies of the same substitutions being observed in naturally evolved proteins, with more frequently substituted sites being selected before less substituted sites. The degree of non-redundancy may be calculated based on the probability of evolutionary resampling at individual amino acid sites between multiple homologs bearing the same substitution based on their overall percent identity relative to the percentage of sites that cannot be substituted due to loss of structure or function, which is about 30%.

In an embodiment, sites for substitution may be prioritized for substitution based on frequencies calculated in successive bins comprising non-overlapping ranges of decreasing percent sequence identity between homologs and the target protein.

In an embodiment, sites for substation may selected wherein sites with complementary hydrogen-bonding groups at ±2 residues in predicted or observed beta-strand structures or at ±3 or ±4 residues in predicted or observed alpha-helical structures are prioritized ahead of sites without such potential hydrogen-bonding partners even if the observed substitution frequencies are lower, but maintaining the substitution-frequency-based priorities within each group.

In an embodiment, a set of mutant proteins harboring increasing numbers of substitutions with progressively decreasing priorities from sequence-based ranking are expressed and evaluated for soluble protein expression. The soluble protein harboring the greatest number of substitutions may be purified and subjected to crystallization screening. Other soluble proteins harboring multiple lysine-to-arginine, aspartate-to-glutamate, or asparagine-to-glutamine substitutions or combinations thereof may be purified and subjected to crystallization screening. In an embodiment, a mutant protein sequence is obtained, and the sequence is synthesized, expressed in an expression system, purified, crystallized, and an x-ray crystal structure may be obtained by x-ray diffraction methods.

This disclosure further relates to a computer-implemented method to automate selection of sites for KR, NQ, or DE mutagenesis based on the frequency of such substitutions in naturally occurring homologs, which can and should tend to avoid sites where lysine is critical for function or structural stability. The disclosure may further relate to a computer-implemented method to predict and characterize the effects of introducing multiple simultaneous KR, NQ, or DE mutations on the thermodynamic stability, solubility and crystallization propensity of proteins.

Crystallization propensity is systematically related to primary sequence, and computational analyses conducted by the inventors have shown that arginine is the most overrepresented amino acid in crystal-packing interfaces in the PDB.

Given the similar physicochemical characteristics of arginine and lysine, aspartate and glutamate, and asparagine and glutamine, the inventors hypothesized that multiple amino acid (AA) substitutions should improve crystallization. To test this hypothesis, computer-implemented methods were developed to rank K/D/N residue sites in a target protein based on redundancy-corrected KR, DE, or NQ substitution frequencies in homologs. The target proteins are naturally occurring proteins refractive to facile high-quality crystallization.

Three unrelated single-domain proteins were tested, one of which readily crystallizes and two of which are recalcitrant to crystallization in their native sequence. It was demonstrated that these proteins tolerated 5-11 KR substitutions with at most minor destabilization, and for two of the proteins, the construct with the largest number of KR substitutions exhibited significantly enhanced crystallization propensity. This approach rapidly produced a 1.9 Å crystal structure of a human protein domain refractory to crystallization with its native sequence. Structures from bulk-KR-substituted domains show that the engineered arginine residues frequently made hydrogen-bonds across crystal-packing interfaces.

These examples demonstrate that introducing multiple KR mutations into a protein, (herein referred to as “Bulk KR substitution”), is a simple and effective method to improve crystallization propensity. Through physicochemical analyses, the disclosers developed and herein present an efficient method for large-scale probabilistic engineering of protein surface properties to improve crystallization, which has been historically considered a stochastic phenomenon refractory to rational experimental manipulation. Therefore, the present disclosure demonstrates that bulk KR substitution represents a rational and efficient method for probabilistic engineering of protein surface properties to improve protein crystallization.

According to certain embodiments, the disclosed methods and systems enable the evaluation of amino acid sequences to predict the likelihood for specific amino acid substitutions to enhance protein crystallization propensity. This capability facilitates higher resolution crystallography and enables a more accurate determination of protein molecular structure.

### DESCRIPTION OF THE FIGURES

Table 1. Summary of expression, stability, and crystallization results for Bulk-KR mutant proteins. The constructs harboring increasing numbers of KR mutations listed in the table also includes all of those in the constructs of the same protein harboring fewer KR mutations. The code “n/p” stands for not possible, while the code “n/a” stands for not applicable or not attempted. The Tₘ and ΔH_{vH} (DH_{vH}) values are labeled as apparent because the reversibility of the unfolding transitions was not assessed in the corresponding thermal denaturation experiments.

Table 2. Crystal-packing contacts in reference and Bulk KR protein structures. Statistics are tabulated separately for backbone (BB) and sidechain (SC) atoms in the indicated amino acids. The abbreviation n/a stands for not applicable. Ratios per engineered arginine reflect exclusively ordered residues and exclude the three disordered arginine residues in the MA_2137-D65R-11 KR structure. Ordered residues are defined as having sufficient electron density to be included in the refined coordinate model deposited in the PDB. Candidate H-bonds were initially identified based on the participating heteroatoms having an internuclear separation ≤3.5 Å, but they were included in the count only if visually confirmed to have reasonable interaction geometry.

Among the five crystal structures analyzed here, only two potential H-bonding interactions in crystal-packing interfaces fulfilled the distance criterion but failed the geometric evaluation. Individual atoms fulfilling the basic distance and geometric criteria with two different potential H-bonding partner atoms were counted as contributing two H-bonds, consistent with Coulomb's law being additive. Atom pairs with internuclear separation ≤4.0 Å not meeting the H-bonding criteria were counted as van der Waals contacts.

Table 3. Data collection & refinement statistics for Bulk-KR crystal structures. Values in parentheses represent statistics for the highest resolution shell.

FIG. 1. Overrepresentation ratios of amino-acids in crystal-packing interfaces compared to overall surface composition in 87,684 crystal structures deposited in the Protein Data Bank (PDB). The thick dotted line schematizes the gain in packing probability produced by K-to-R mutations, while the thin dotted lines schematize the changes from N-to-Q and D-to-E mutations. The overrepresentation ratios are segregated by protein 2° structure as assessed by DSSP, and the amino acids are ordered in decreasing order of overrepresentation ratio in α-helical secondary structure.

FIG. 2. According to one embodiment, FIG. 2 is a representative output from the inventor's Bulk-R computer-implemented method to analyze lysine-to-arginine substitution patterns in homologous proteins. Results are shown for the hPDIa domain. All sequence analyses are conducted on sets of proteins spanning 10% ranges (bins) of sequence identity relative to the target protein, with the exception of the highest identity bin which spans 90-99% identity to avoid including mutant protein sequences. (a). The top graph shows the Shannon entropy for each lysine site in the target protein in the indicated mutually exclusive sequence % ID bin. The middle graph shows the fraction of residues other than lysine, while the bottom graph shows the ratio of arginine/lysine residues at those sites in each indicated sequence identity bin. (b). The total number of sequences in each mutually exclusive % ID bin in which arginine is observed at a lysine site in the target protein. (c). A heuristically redundancy-reduced count of the number of arginine substitutions in each of the bins based on the algorithm described in the Methods section. (d). The expected number of independent substitutions of arginine for lysine in the course of evolution between up to the seven most diverged sequences in each of the indicated % ID bins. The graph in panel (c) displays the results of the first redundancy-correction calculation described in the text, while the graph in panel (d) displays the results of the second, which is generally used to rank lysine sites in the target protein for mutagenesis. (e) Table showing the rankings to illustrate the site-selection algorithm. The final column reports the expectation value for the number of independent substitutions between all pairs of sequences in the indicated percent identity bin, while the total and redundancy-reduced counts are reported relative to the target sequence.

FIG. 3. Thermodynamic stability of bulk-KR mutants characterized using thermal denaturation experiments monitored by circular dichroism (CD) spectroscopy. Experiments were conducted using protein samples at 2 mg/ml scanned at a rate of 3°/min in a buffer containing 100 mM NaCl, 10 mM Tris-Cl, pH 7.5, with the addition of 1 mM SAH for the MA_2317 constructs. The suppression of the low-temperature transition in the hPDIa-9KR construct could be attributable to an intra-helical salt bridge between the sidechains of residues E62 and R65. The latter residue is one of the KR mutations in this construct not shared by the hPDIa-7KR construct, and the sidechain of residue K65 does not make any H-bonds at all in the crystal structure of the multidomain construct (PDB id 4EKZ). Therefore, the sidechain salt-bridge produced by the K65R mutation could potentially stabilize local structure in the hPDIa domain.

FIG. 4. Crystallization hit counts from the 1536-condition high-throughput automated microbatch-under-oil crystallization screen at the National Crystallization Center at the Hauptman-Woodward Institute (HWI). These screens were conducted using generation 19 of the HWI crystallization cocktail collection. The proteins were at ~15 ml/ml in the stock solutions used for screening, which contained 100 NaCl, 10 mM DTT, 10 mM Tris-CI, pH 7.5. The screening plates were maintained at 4° C. for the first week and then moved to 25° C. for the remainder of the screening period.

FIG. 5. Crystal-packing interactions in structures from protein constructs containing bulk-KR mutations and the parental protein constructs. The protein backbone is shown in ribbon representation, colored in different shades of black and gray for symmetry mates and for the subunits modeled in the asymmetric unit of each crystal structure. The sidechains of the native arginines and residues mutated from lysine-to-arginine are shown in space-filling representation on the left and in stick representation in the zoomed-in views of local packing interactions in the boxes to the right. The residues in the hPDI (abb‘xa’) and MA_2137-D65R structures are the native lysine residues, while the residues in the hPDIa-9KR and MA_2137-D65R-11 KR structures are the residues mutated to arginine. Asp, asn, glu, and gin residues making H-bonds to the illustrated lysine and arginine residues in the boxes are shown in stick representation, while the backbone atoms making H-bonds to those residues are shown in ball-and-stick representation.

FIG. 6. Pairwise percent sequence identity distributions between homologs in mutually exclusive bins spanning 10% ranges of sequence identity to target protein hPDIa.

FIG. 7. Stereo views of crystal-packing interactions in structures from the parental proteins and protein constructs containing bulk-KR mutations. The protein backbone is shown in ribbon representation, colored lighter gray for symmetry mates and darker grey for the subunits modeled in the asymmetric unit of each crystal structure. The sidechains of the native arginines and residues mutated from lysine-to-arginine are shown in space-filling representation. Note that the residues in the parental hPDI (abb‘xa’) and MA_2137-D65R structures on the left are the native lysine residues, while the residues in the hPDIa-9KR and MA_2137-D65R-11 KR structures on the right are the residues mutated to arginine.

FIG. 8. Crystal structures of Bulk-KR-substituted protein constructs show no significant conformational or stereochemical changes compared to reference structures. (a) Comparison of our crystal structure of hPDIa-9KR (FIG. 5b and Table 3) to an earlier structure of the same domain as part of a much larger multi-domain hPDI (abb‘xa’) construct (PDB ID 4EKZ). (b) Comparison of our crystal structures of MA_2137-D65R-11KR (FIG. 5d and Table 3) and MA_2137-D65R (PDB ID 6MRO). The loop at residues 141-152 is ordered by a high concentration of Ca++ present in the mother liquor for the latter structure, while the was no Ca++ present in the mother liquor for former structure. Least-squares alignments were performed in PyMOL (www.pymol.org) and ChimeraX using the default algorithms. (c-e) More detailed analyses of the structural differences in the Bulk KR constructs compared to the parental domains were performed using the Global Distance Test (GDT). The top graphs in panels c-d show the Ca displacements between all shared residues in the alignments used for the test, with the vertical dotted lines indicating the sites of the KR mutations. The bottom graphs in these panels show the corresponding histograms of the Ca displacements for all residues and the engineered arginine residues (red). Panel e shows the fraction of Ca atoms within the indicated cutoff distances as calculated by the High Accuracy GDT algorithm.

FIG. 9. Solubility assays on selected hPDIa and MA_2137 constructs. Assays were conducted at room temperature. The lower graph in the center column shows data collected without S-adenosylhomocysteine (SAH) in the buffer, which is a product of the methyltransferase reaction catalyzed by MA_2137, while all of the other solubility assays on this protein were conducted in the presence of 1 mM SAH. The top graphs in both MA_2137 columns show the same data, which are duplicated but matched in scale to the graphs beneath them in order to provide a visual reference.

FIG. 10. Optical micrographs showing time evolution of representative MA_2137-D65R crystallization screening reactions showing evidence of liquid-liquid phase separation. Representative micrographs from the HWI generation #19 1,536 well crystal screen (https://hwi.buffalo.edu/crystallization-cocktails/) show evidence of liquid-liquid phase separation for MA_2137-D65R. Other than a tag and 12-residue internal loop, likely to be a calcium binding site (FIG. S3b), this protein is ordered. The cocktail recipes are as follows for the individual wells: #5: 0.1 M Na2S2O3, 0.1 M Tris, pH 8, 20% (w/v) PEG 4000; #6: (HR SaltRx Screen HT A12) 3.2 M NaCl, 0.1 M NaOAc, pH 4.6; #7: (HR SaltRx Screen HT D4) 6.0 M NH4NO3, 0.1M NaOAc, pH 4.6; #8: 0.1 M LiBr, 0.1 M Tris, pH 8, 20% (w/v) PEG 4000.

FIG. 11. Comparison of overrepresentation ratios of amino acids in crystal-packing interfaces in our analysis of 87,684 crystal structures 38 (FIG. 1) to an earlier analysis of contact potentials in crystal-packing interfaces in 233 crystal structures.

### DETAILED DESCRIPTION

The use of the term “about” herein means±20% of a stated value.

The first phase of this research leading to the present disclosure identified a large number of local primary sequence patterns, called crystallization epitopes, which were strongly overrepresented in crystal-packing interfaces.[2] It was demonstrated that introducing these epitopes individually into proteins generally increases their crystallization propensity, and introducing multiple such epitopes progressively increases crystallization propensity. The cumulative nature of the observed improvements suggested that multiple simultaneous mutations could potentially produce definitive improvements in crystallization propensity in a single protein construct based on large-scale probabilistic engineering of protein surface properties. Therefore, according to certain embodiments, the present disclosure relates to an efficient method to produce definitive improvements in crystallization propensity in a protein construct based on probabilistic engineering of protein surface properties while preserving protein stability and solubility.

The present disclosure relates to rational methods to improve protein crystallization properties accounted for sequence and structural analyses of crystallization results and associated thermodynamic properties. The inventor's determined that several protein surface properties can function as a significant determinant of protein crystallization propensity. It was also demonstrated that overall thermodynamic stability was not a major determinant of protein crystallization propensity.

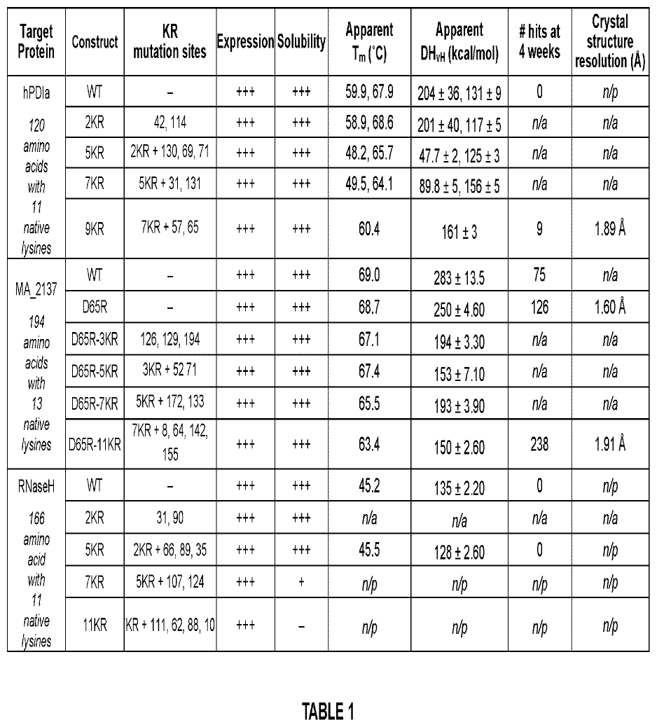

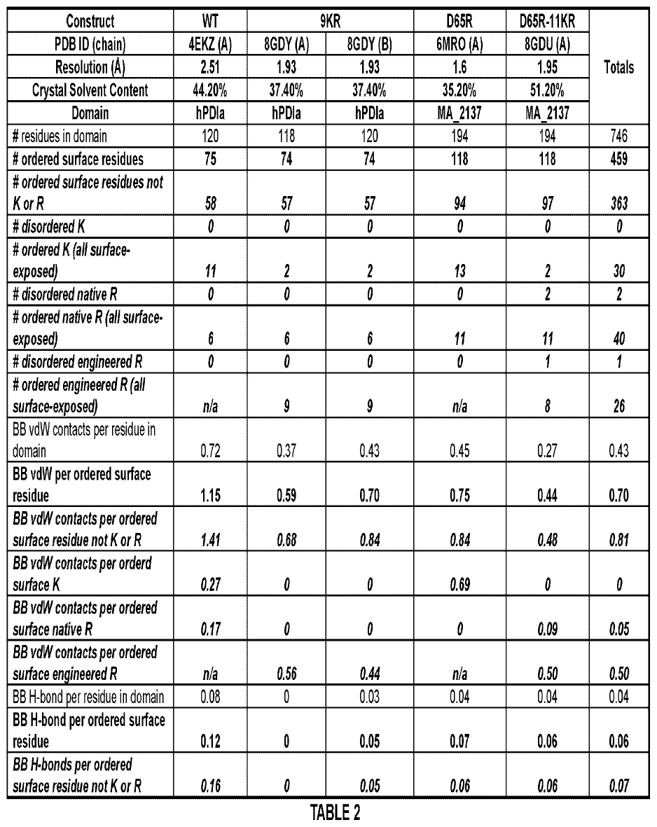

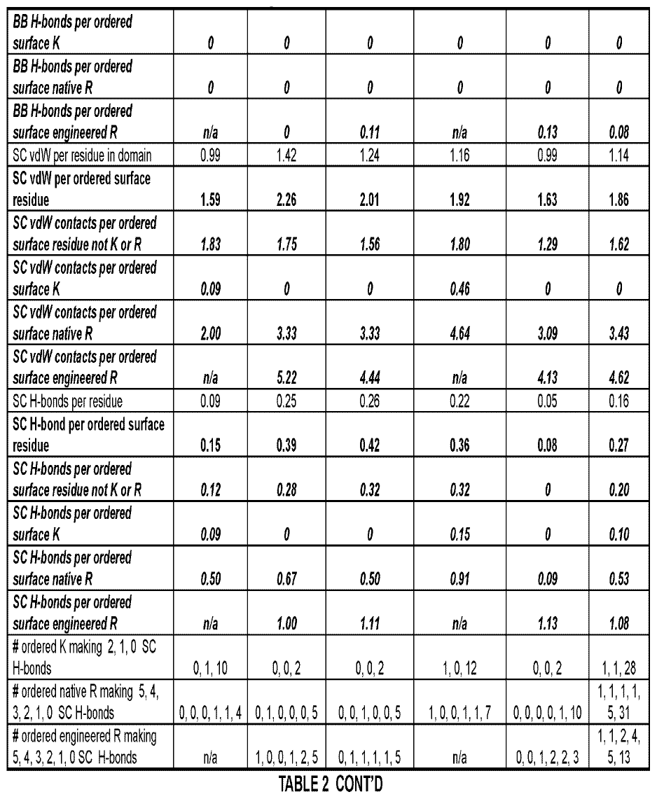

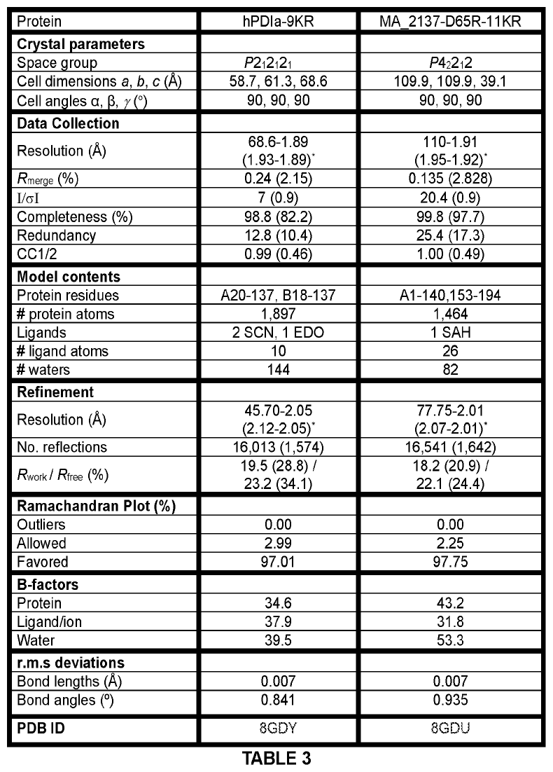

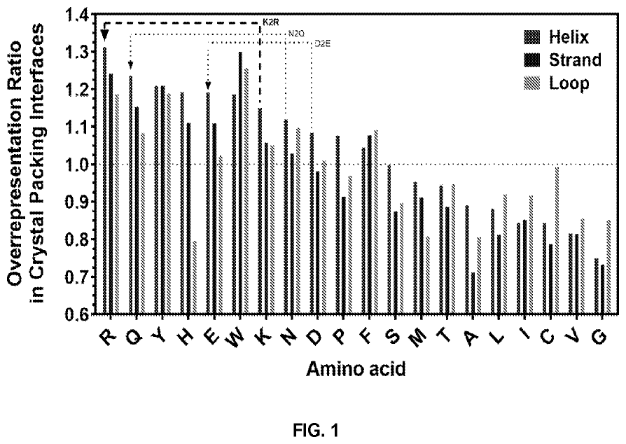

The inventors identified a number of primary sequence properties that correlate with successful crystal structure determination, including significant anticorrelations with predicted backbone disorder and the sidechain entropy of predicted solvent-exposed residues as well as significant positive correlations with the fractional content of several individual amino acids. Further, 87,683 crystal structures from the Protein Data Bank (PDB) were analyzed and contiguous amino acid patterns were identified strongly overrepresented in crystal packing interfaces. This analysis also generated data on the relative overrepresentation of individual amino acids in crystal-packing interfaces segregated by secondary structure (FIG. 1).[3]

Computational analysis of crystal-packing interactions in the PDB showed a substantially higher probability for arginine to mediate inter-molecular packing contacts than lysine (FIG. 1), consistent with analyses of correlations between primary sequence features and protein crystallization propensity. The observation that arginine mediates crystal-packing contacts more frequently than lysine is particularly notable because the entropy of the arginine sidechain is estimated to be somewhat higher than that of lysine, which implies its immobilization in an intermolecular interface should tend to incur a higher entropic penalty that reduces its probability of making crystal-packing contacts. Therefore, the more frequent occurrence of arginine compared to lysine in crystal-packing contacts suggests that the guanidino group on arginine is substantially “stickier”, in terms of intermolecular interaction free energy, than the primary amine on lysine.

One straightforward contributor is likely to be the greater hydrogen-bonding (H-bonding) potential of the guanidino group in arginine compared to the primary amine in lysine (i.e., the presence of 5 H-bond donor protons compared to 3). The significance of this factor is supported by molecular dynamics simulations showing multiple stable interaction geometries for salt bridges containing arginine. Arginine is much more effective than lysine in inhibiting protein aggregation, which is believed to reflect strong solvation interactions between arginine and protein surfaces, including at apolar sites. The guanidinium ion, a close analog of the guanidino group in arginine, shows thermodynamically significant interactions with some proteins in their native conformations, and it also enhances the solvation and solubility of apolar groups.

The greater interaction potential of arginine is further supported by the properties of the guanidinium ion as a potent protein denaturant, a property that is not shared by primary amines. The thermodynamics of the denaturation process involve an interplay between enthalpically favorable solvation interactions with protein group, perturbations in water structure that weaken the hydrophobic effect, and competition with polar groups on the protein for H-bonding to water.

Experimental measurements of the free energy of transfer of model peptides support strong solvation of the backbone being a dominant contributor to guanidinium-induced protein denaturation. NMR measurements of backbone amide exchange rates show that guanidinium does not H-bond to the backbone, while quantum chemical calculations support its favorable solvation of the backbone deriving from strong enthalplic interactions with the Cβ atom of the amino acids. Finally, molecular dynamics simulations show stabilizing interactions increasing the duration of non-covalent associations of guanidinium with both the backbone and many sidechains.

Based on the multifaceted evidence that arginine mediates stronger intermolecular interactions with protein groups than lysine, the present inventors hypothesized that introducing multiple arginine-to-lysine (KR) substitutions in a protein would enhance crystallization propensity. It was furthermore hypothesized that, given the very similar physicochemical properties of arginine and lysine in terms of size and polarity, multiple simultaneous substitutions would be tolerated without significantly impairing thermodynamic stability. The present disclosure demonstrates the validity of these hypotheses.

### KR Mutation Site-Selection Algorithm and Software

In certain embodiments, a bulk amino acid substitution crystallization propensity application includes an amino acid mutation site-selection module and a graphical user interface (GUI) module. The provided application can rank amino acid substitutions based on the frequency of these substitutions in naturally evolved sequences in a phylogenetic alignment thereby allowing for ranking of possible substitutions based on their propensity to increase crystallization in a target protein engineered to contain such substitutions.

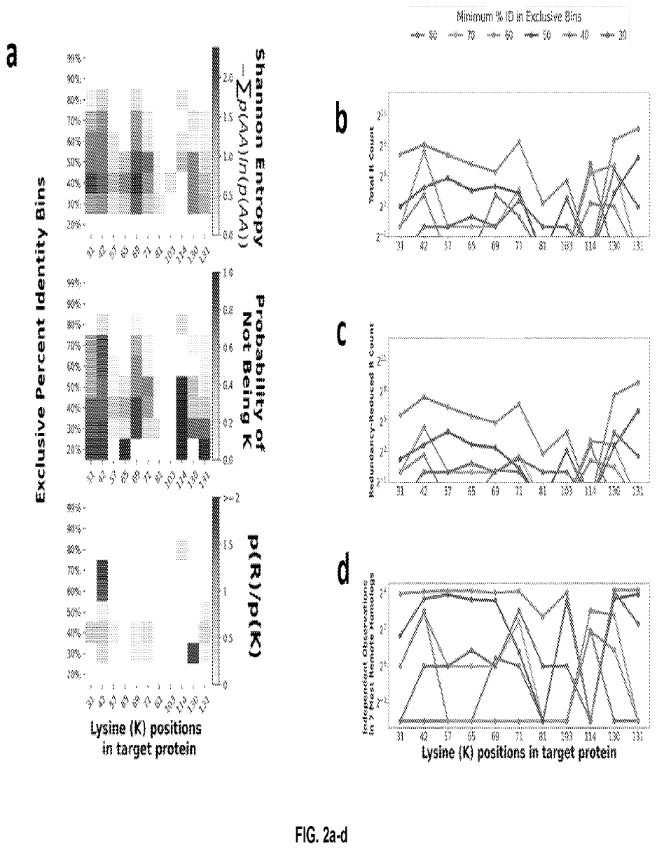

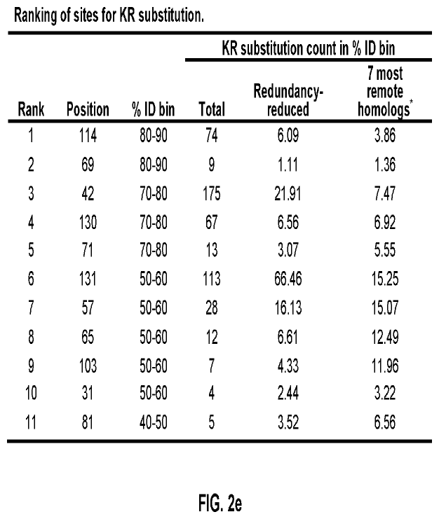

For example, sites for Bulk KR substitution can be ranked and selected based on the frequency of these substitutions in naturally evolved sequences in a phylogenetic alignment (FIG. 2). The procedure can be fully automated in Python or other forms of computer code and can also be run interactively via local or remote servers or on the worldwide web.

In some embodiments, the present disclosure relates to a computer implemented method employing an algorithm. The algorithm implemented by the program can rank sites based on a redundancy-compensated estimate (explained below) of the frequency of KR substitutions in homologous sequences, which are divided into mutually exclusive bins with progressively lower levels of overall percent identity relative to the target sequence. As seen in FIG. 2, the first bin can include sequences with less than 99% identity (to avoid mutant variants of the target sequence) and greater than or equal to 90% identity. The next bin can include sequences with less than 90% identity and greater than or equal to 80% identity, while subsequent mutually exclusive bins can reduce the range of identity levels in 10% steps down to a minimum of 30%. The algorithm can step through these bins in order from highest to lowest percent identity. In each bin, it can select first the site with the highest estimated number of independent arginine substitutions among the most remotely related homologs in that bin (FIG. 2d). It can then select additional sites that show lysine-to-arginine substitutions in the same percent identity bin in decreasing order of their estimated number of independent arginine substitutions among the most homologs, stopping when it hits a user-adjustable minimum count.

This threshold count can be imposed to avoid selecting a site based on an arginine substitution in a single sequence that could potentially be inaccurate or present only in a small number of very closely related sequences that could potentially share a function-impairing or stability-impairing mutation. The threshold count can default to a value of 1.1, which ensures observation of a lysine-to-arginine substitution in at least two sequences with no more than ~93% identity to one another. Given the details of heuristic sequence-divergence metric described in the Methods, every 0.1 increase in the threshold count is equivalent to requiring ~7% lower identity in two sequences having the substitution or an additional pair of homologs at the same divergence level having the substitution.

After hitting the specified threshold or selecting all sites showing arginine-to-lysine substitutions above the threshold count in the bin being evaluated, the algorithm can progress to the next lower percent identity bin and implement the same site-selection protocol in that bin. This selection algorithm can continue until the same site-selection protocol is executed in the final mutually exclusive bin, which can include sequences with less than 40% identity and greater than or equal to 30% identity to the target protein. The algorithm can thus provide a rank-order for mutation of all lysine sites in the target protein at which mutations to arginine are observed above the threshold count in any of the evaluated percent identity bins. The program can output the complete list of sites selected by the algorithm ranked according to their order of selection (e.g., as shown in FIG. 2e).

In certain embodiments, the computer implemented method, GUI module, and software performing said method can provide graphical displays of results of calculations of the Amino Acid Mutation Site-Selection module such as summary parameters characterizing the amino acid distribution in the homologs in each of the percent-identity bins at every lysine site in the target sequence (FIG. 2), as well as a graphical display of the overall sequence diversity in each of the bins (FIG. S1). The displayed summary parameters can be the results of the Amino Acid Mutation Site-Selection module such as the Shannon entropy of the amino-acid frequency distribution, the frequency of all residues other than lysine, the arginine-to-lysine ratio, the total count of sequences with an arginine residue at the site, and two different estimates of that count after compensation for redundancy between those sequences.

The Amino Acid Mutation Site-Selection module can perform at least two redundancy-compensation calculations (redundancy-compensated estimated counts). For example, it can use a heuristic estimate of the degree of mutational resampling between pairs of sequences that is described in the Methods section along with explanations of the details of that algorithm and a second algorithm summarized below. In brief, the heuristic estimate of the degree of mutational resampling between pairs of sequences can evaluate all sequences using a calculation that has rigorously correct behavior in the cases of full redundancy and full independence between the sequences but is otherwise approximate. The second redundancy-compensation calculation can provide a rigorous probabilistic estimate of the number of independent observations in the seven most remotely related sequence pairs in the bin having arginine at that site. Extending this calculation to more sequences can be performed with enhanced computational resources; however, obtaining an estimate based on a limited set of the most diverged homologs provides a highly effective method to ensure that multiple independently determined protein sequences have an arginine residue at the lysine site in the target protein, which is the essential goal of the redundancy-compensation calculations. This second calculation is generally used for the automated site-ranking algorithm described above.

The Amino Acid Mutation Site-Selection module can additionally provide a ranking of sites for introducing aspartate-to-glutamate and asparagine-to-glutamine mutations together with a record of which of those sites have potential ionic interaction or H-bonding partners in the target sequence that would tend to reduce the entropy of the longer sidechains in beta-sheet (i±2) or α-helical (i±3,i±4) secondary structures. (The rationale behind this approach is described in the Discussion section below.) Lysine, arginine, and histidine are considered potential ionic interaction partners for glutamate and H-bonding partners for glutamine. Asparagine, glutamine, serine, and threonine are considered potential H-bonding partners for glutamate and glutamine with the addition of aspartate and glutamate for glutamine.

The site-selection strategy according to the present disclosure, which is based on making amino acid substitutions observed in multiple non-redundant homologs, can tend to preserve activity because evolutionary selection for organismic fitness tends to preserve protein function. Consistently with the observed plasticity in homologous protein sequences in the course of biological evolution, large-scale saturation mutagenesis studies using next-generation sequencing methods support most amino acid substitutions at most positions preserving qualitative protein function. Based on these data, physiochemically conservative substitution observed in strongly homologous proteins are unlikely to abrogate activity. Nonetheless, multiple conservative point mutations could impair protein function, so direct experimental evaluation of protein activity would be required to verify the functional competency of proteins harboring Bulk KR mutations. Lysine residues known or suspected to be important for activity or involved in oligomeric interactions should generally be avoided when implementing the method.

### Test Protein Selection and Expression

The bulk amino acid substitution approach was tested using three proteins with different crystallization properties. Substitutions evaluated were KR, NQ, or DE substitutions. Discussions herein of “KR mutations” are exemplary and generally also apply to NQ or DE substitutions.

The hPDIa domain is a human drug target that represents the first of four domains in the endoplasmic-reticulum-resident human Protein Disulfide Isomerase (hPDI) protein.[4],[5] The hPDIa domain had never successfully been crystallized on its own, but its structure was known from a relatively low-resolution crystal structure of a much longer multi-domain construct containing hPDIa 88, which enables evaluation of the impact of Bulk KR substitutions on its structure, as reported below.

E. coli RNaseH is difficult to crystallize in the absence of ligands stabilizing active site structure but has had its crystal structure determined by groups studying its enzymological mechanism and folding.[6]

MA_2137, an S-adenosyl-methione-dependent RNA methyltransferase from Methanosarcina acetivorans,[7] crystallizes well in the presence of S-adenosyl-homocysteine (SAH), the product of the methyltransferase reaction that it catalyzes. MA_2137 was included because increasing hit count in high-throughput crystallization screening is strongly correlated with the probability of successful crystal-structure determination, which implies quantification of hit count for a protein that crystallizes relatively easily is an effective assay for crystallization propensity. KR mutations were introduced into the D65R mutant of MA_2137 because this single mutation improves crystallization of this protein and, therefore, this protein was a good candidate to test whether bulk KR substitutions could further improve crystallization.

The inventors introduced 2 to 13 KR mutations into these proteins (Table 1), and examined the expression and solubility levels of the full set of mutant constructs when expressed from a pET plasmid using T7 RNA polymerase in E. coli, which yielded high-level expression of the three parental proteins in the form of efficiently purified monodisperse monomers. The largest number of KR mutations tested preserved high-yield protein production in a monodisperse state for both hPDIa and MA_2137-D65R (i.e., the hPDIa-9KR and MA_2137-D65R-11 KR constructs). Only two native lysine residues remained in each of these constructs. The RNaseH-2KR and RnaseH-5KR constructs similarly preserved high-yield protein production in a monodisperse state. However, the RnaseH-7KR construct yielded polydisperse protein that co-purified with the Hsp33 molecular chaperone protein, while the RnaseH-11 KR was completely insoluble even though it expressed at a high level. The stability studies presented in the next section confirmed that RnaseH has a low thermal melting temperature of ~45° C., making it marginally stable, which likely explains its tolerance for fewer KR mutations than the other target proteins.

### KR Mutations are Generally Only Minimally Destabilizing

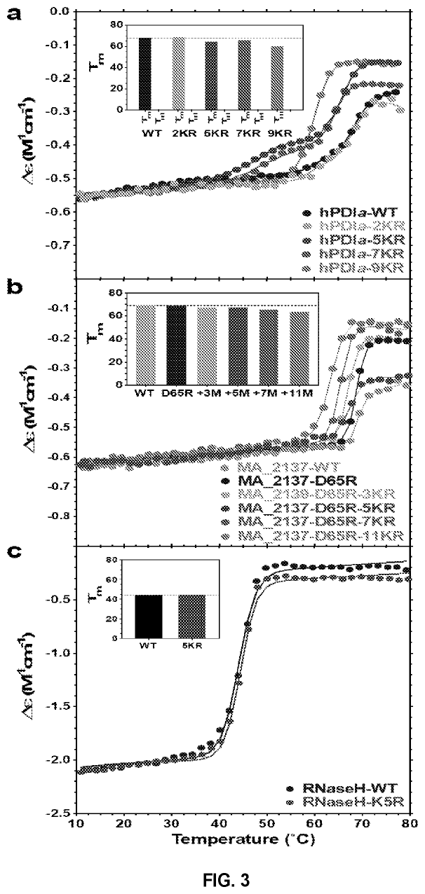

The thermal stabilities of all the successfully purified bulk KR construct were characterized using circular dichroism spectroscopy. These assays showed a variable but generally very small degree of destabilization by KR mutations (FIG. 3 and Table 1). RNase-5KR showed an approximately unaltered apparent Tₘ compared to the wild-type protein, demonstrating that KR mutations can have a completely neutral effect on stability. PDIa-9KR showed an ~8° reduction compared to the 68° C. apparent Tₘ of the wild-type domain, while MA_2137-D65R-11 KR shows an ~6° reduction compared to the 69° C. apparent Tₘ of the parental protein. Considering the entire set of mutant proteins in the study that could be purified, which included 25 different KR mutations (Table 1), there was on average a 0.54+/−0.30° reduction in apparent Tₘ per KR mutation. Therefore, KR mutations are generally very well tolerated, although large sets of mutations tend to produce modest reductions in protein stability, which can reduce soluble protein yield in vivo when the stability of the wild-type (WT) protein is relatively low.

### Bulk KR Mutations Enhance Crystallization Propensity and Yield Strongly Diffracting Crystals

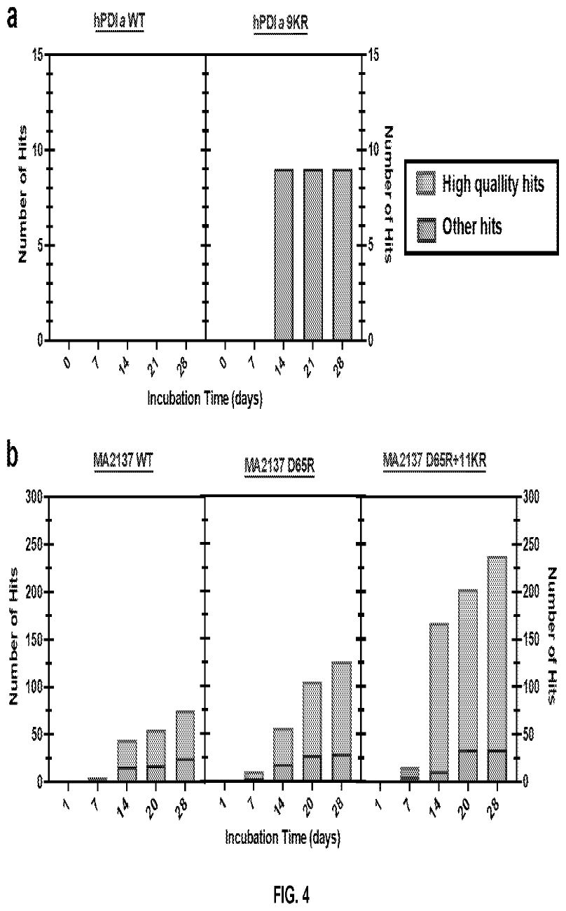

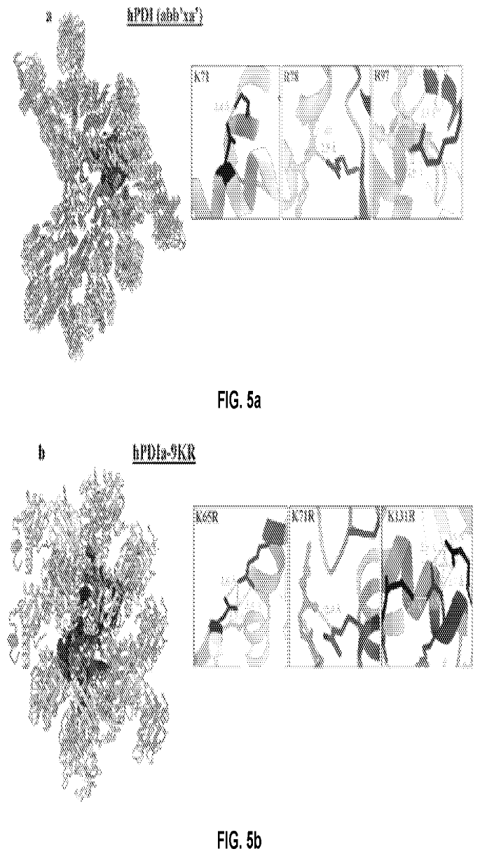

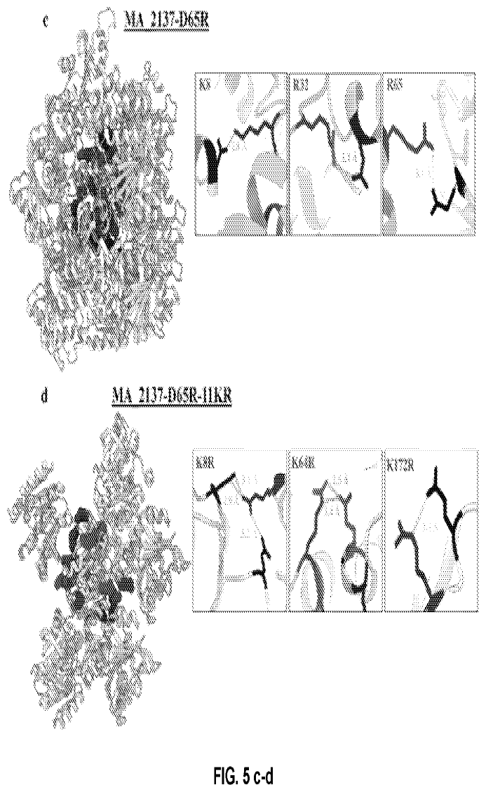

The purified protein constructs harboring the largest number of KR mutations (i.e., PDIa-9KR and MA_2137-D65R-11 KR) along with matched controls were screened for crystallization at the National Crystallization Center at the Hauptman-Woodward Institute™ (HWI) using their automated, high-throughput 1536-condition screen.[8] This microbatch-under-oil screen was employed for initial crystallization screening by the Northeast Structural Genomics Consortium™, (nesg.org), which used it to generate 664 crystal structures deposited in the PDB. Neither the WT or 5KR construct of RNaseH yielded any crystallization hits in a screen intentionally conducted without any ligands stabilizing active site structure in order to provide the most exacting test of protein crystallization propensity; the lack of success for this protein was potentially influenced by the high 15 mg/ml protein concentration used for screening, which produced pervasive amorphous precipitation in the screen at the earliest observation times. However, the hPDIa-9KR and MA_2137-D65R-11KR constructs both yielded significantly more crystallization hits than the control proteins. MA_2137-D65R-11KR yielded hits under twice as many conditions as the MA_2137-D65R control protein, while hPDIa-9KR yielded 9 high quality hits compared to no hits at all for the WT construct (FIG. 4 and Table 1). A small number of hit conditions for each protein were chosen for optimization, which very rapidly yielded 1.9 Å structures for both Bulk KR constructs based on a single session of remote synchrotron diffraction screening and data collection (FIG. 5 and Table 3). Therefore, for both target proteins, crystallization screening only had to be conducted on the soluble construct harboring the largest number of Bulk KR mutations in order to rapidly obtain high-quality crystal structures.

### Bulk KR Mutations Perturb Protein Structure and Frequently Make H-Bonds in Crystal-Packing Interfaces

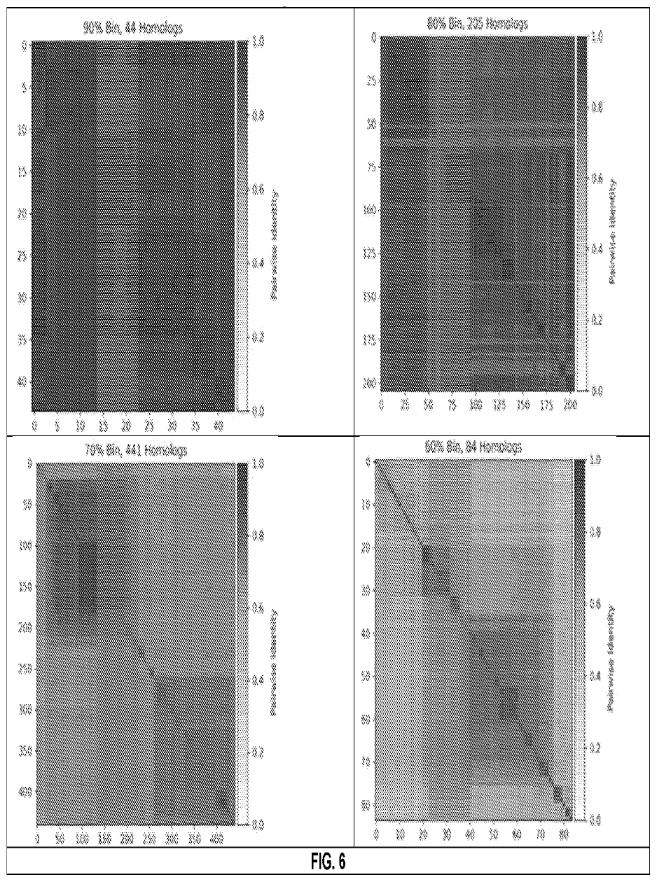

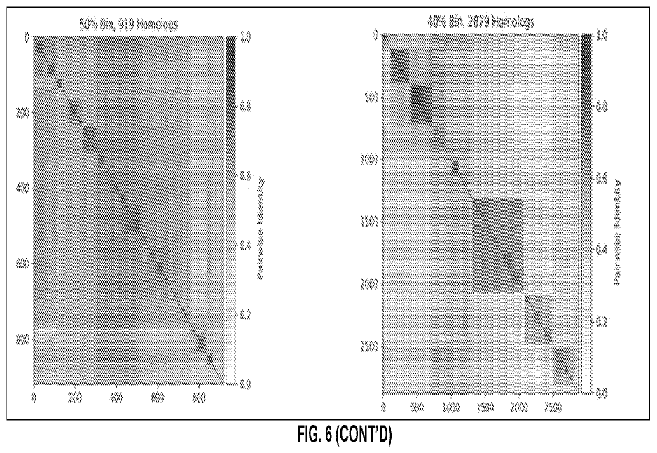

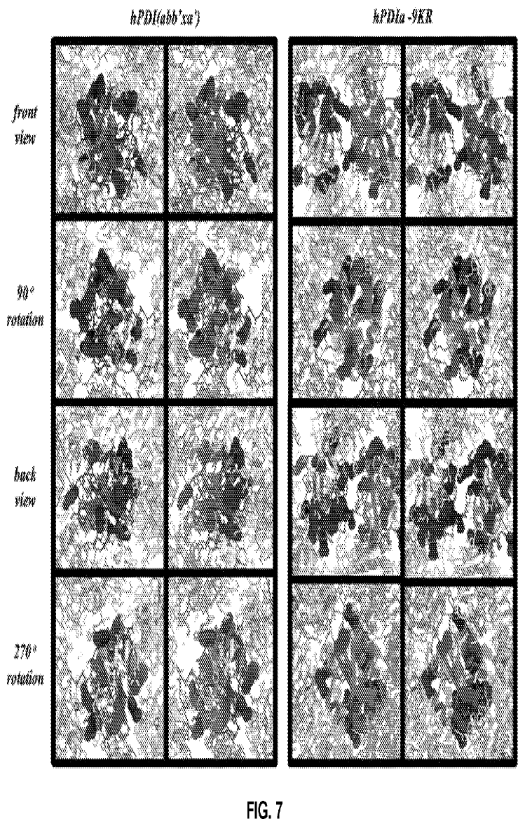

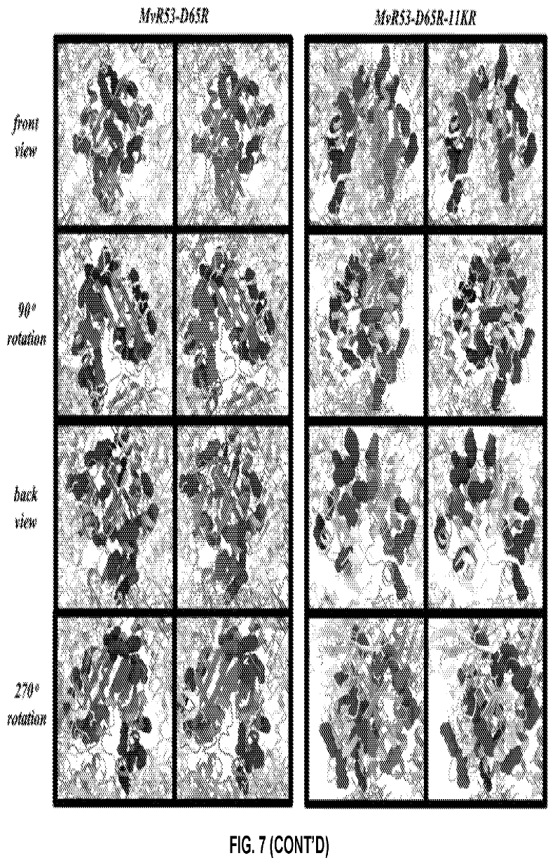

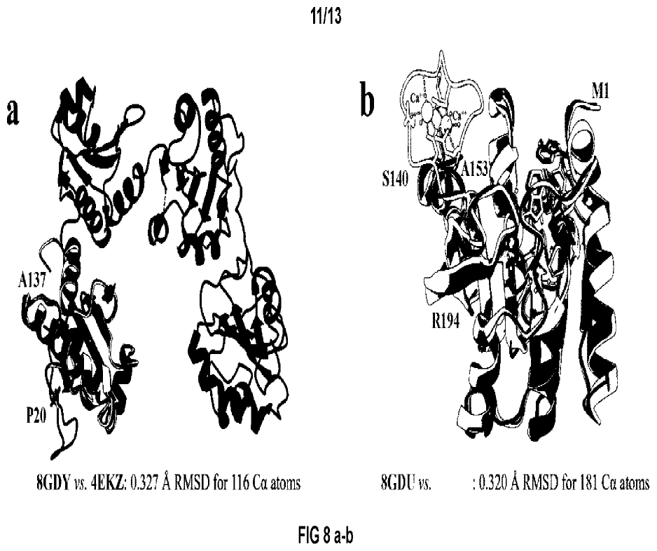

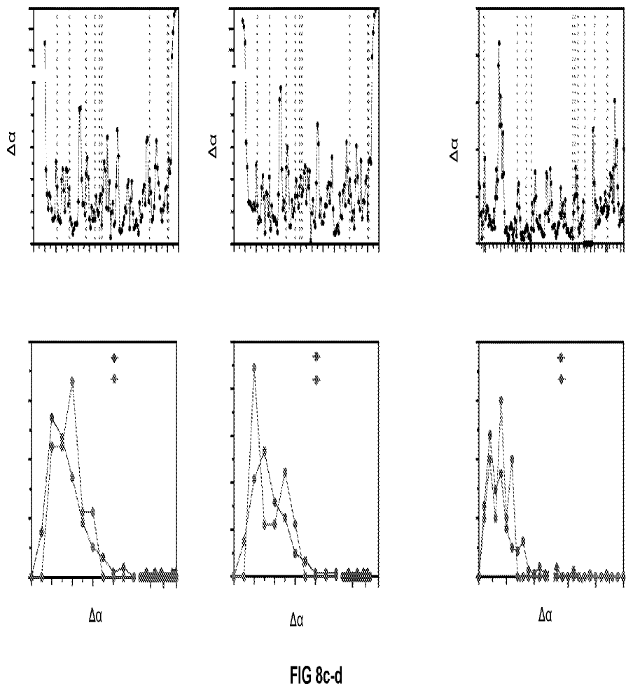

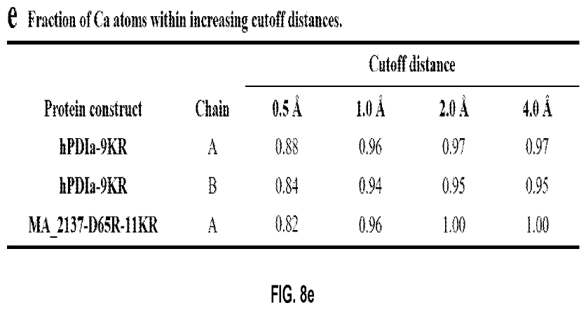

The 1.9 Å crystal structures of the Bulk KR substitution (FIGS. 5-6 and Table 3) constructs showed 0.32-0.33 Å root-mean-square deviations for their backbone Ca atoms compared to the reference structures (FIG. 8) (i.e., the much larger multidomain hPDI (abb‘xa’) construct for hPDIa because the isolated domain has never successfully been crystallized before and the parental MA_2137-D65R construct for MA_2137-D65R-11 KR). The observed deviations are close to the expected coordinate error in well-refined crystal structures in the operative resolution range,[9] indicating that the disclosed Bulk KR substitution method does not significantly perturb protein conformation for either of the targets. Moreover, the detailed analyses of protein conformation presented in FIG. 8 demonstrate that the distribution of the local backbone deviations at the KR mutation sites is equivalent to that observed for all residues in each structure.

Detailed analyses of the intermolecular interactions in the crystal structures demonstrated that the engineered arginine sidechains made extensive crystal packing contacts, substantially exceeding the number of van der Waals contacts and especially H-bonds made by the native arginine sidechains in the same constructs and greatly exceeded the number of both kinds of contacts made by lysine sidechains in the parental constructs (Table 2 and FIG. 5). The larger number of crystal-packing contacts made by the engineered vs. native arginine residues could potentially reflect greater sequestration of the native residues in local surface interactions reducing the probability of reaching across a packing interface to make an energetically stabilizing interaction with a neighboring molecule in the crystal lattice. The observed trends support the premise underlying the Bulk KR substitution strategy, which was based on the substantially stronger overrepresentation of arginine vs. lysine in crystal-packing interfaces in the large-scale analysis of crystal structures previously deposited in the PDB (FIG. 1).

On average, the well-ordered engineered arginine sidechains in the Bulk KR structures make 1.08 H-bonds each to a neighboring protein molecule in the crystal lattice, compared to 0.48 each for the native arginine residues (Table 2). In comparison, the well-ordered lysine sidechains make an average of 0.10 H-bonds each to a neighboring protein molecule in the native structures and none in the Bulk KR structures. These results support the physicochemical basis of the greater overrepresentation of arginine compared to lysine in crystal-packing interfaces in the PDB being attributable to the guanidino group in arginine being substantially more efficacious than the primary amine group in lysine in mediating energetically stabilizing H-bonds in the relevant stereochemical contexts (FIG. 5).

The number of van der Waals contacts per ordered sidechain follows a similar trend. Engineered and native arginine residues each make on average 4.62 and 3.43 contacts, respectively. In contrast, lysine residues in the native structures each make on average 0.28 contacts, while the lysine residues remaining in the engineered structures make none (Table 2). The greater number of intermolecular van der Waals contacts made by the arginine sidechains could potentially be influenced by their greater H-bonding propensity leading to more frequent occurrence in crystal-packing interfaces. Notably, the number of backbone H-bonding and van der Waals interactions made by the arginine vs. lysine residues in the reference and engineered structures do not show any clear trends (Table 2).

### Influence of Bulk KR Mutations on Protein Solubility in PEG3350 Solutions

Thermodynamic solubility assays using polyethylene glycol 3350 (PEG3350) to induce protein precipitation can assess the relative free energy of the hydrated state of individual protein molecules compared to the most favorable self-associated state under conditions of constant ionic strength but reduced water activity (effective water concentration). In practice, these assays monitor optical density at 280 nm in the supernatant of solutions containing different concentrations of protein in the presence of increasing concentrations of PEG3350 after centrifugation to remove large particulate molecular assemblies. So, the assays effectively measure the equilibrium concentration of protein that remains soluble as water activity is reduced. The observed results depend intrinsically on the free energy of the self-associated state, which varies significantly for different proteins in different solvent environments and can include crystalline phases and liquid-liquid phase separated (LLPS) phases in addition to heterogeneous amorphously precipitated phases. This factor can complicate interpretation of thermodynamic solubility assays, but they nonetheless provide insight into physicochemical properties that ultimately control protein crystallization behavior.

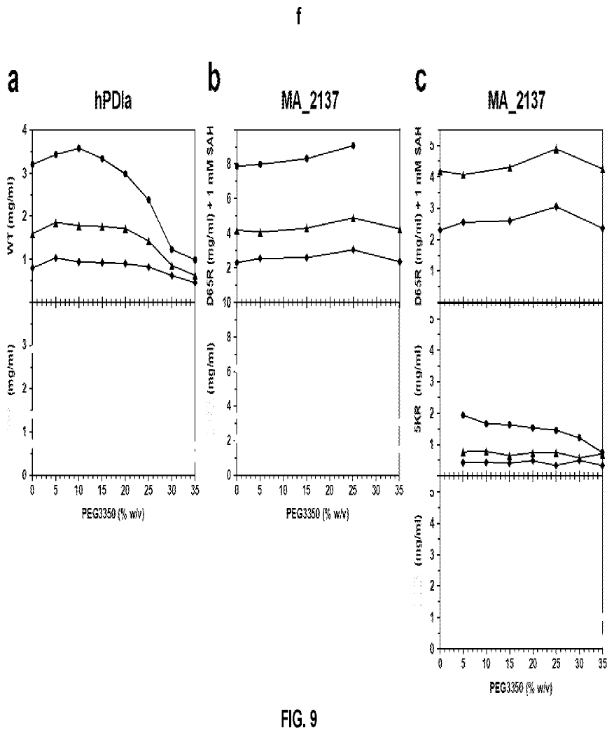

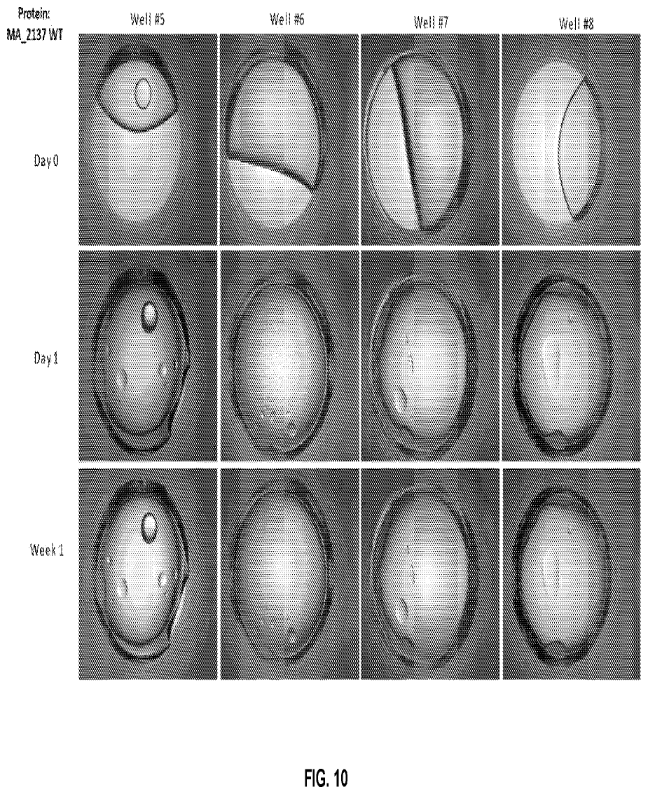

PEG3350 precipitation assays on WT hPDIa and the 9KR mutant show no significant difference in their behavior in PEG3350 precipitation assays (FIG. 9a), indicating that the Bulk KR substitutions in this protein domain do not alter its thermodynamic solubility under these conditions even though they enable crystallization and high-resolution structure determination of a domain that does not crystallize at all with its native sequence. Even when harboring the 9KR mutations, hPDIa crystallizes only under a very small fraction (0.6%) of the solution conditions explored in high-throughput crystallization screening (FIG. 4) while showing amorphous precipitation in many of them. Therefore, the thermodynamic solubility assays on the hPDIa constructs are likely measuring the free energy of the hydrated state of individual protein molecules compared to amorphously precipitated phases, and they demonstrate that the physicochemical properties controlling the formation of such phases are likely different from those controlling protein crystallization behavior.

PEG3350 precipitation assays on our MA_2137 constructs demonstrate more complex phase behavior (FIG. S4b-c) likely reflecting different physical forms of self-association under assay conditions. Notably, the WT and D65R mutant could not be precipitated by the highest 35% (v/v) concentration of PEG3350 that was assayed (FIG. S4b). These protein constructs instead showed some tendency to exhibit a small increase in optical density at low PEG3350 concentration, likely reflecting light scattering due some form of protein self-association in a low-density state that does not sediment during low-speed centrifugation. During crystallization screening, these constructs showed clear evidence of liquid-liquid phase separation LLPS without any apparent amorphous precipitation in many reaction conditions (FIG. S5). Therefore, the inability to precipitate these constructs at high PEG3350 concentration likely reflects LLPS being energetically more favorable for this protein under conditions of low water activity than amorphous precipitation.

The crystal structures of MA_2137 constructs showed clear and well-ordered electron density for every residue in this 202-residue protein except for: (1) the C-terminal hexahistidine tag that was added to enable purification using NiNTA affinity chromatography; and (2) a 12-residue internal loop that is disordered in the MA_2137-D65R-11 KR structure although well ordered by Ca++ ions from the mother liquor in the structure of the parental MA_2137-D65R construct (FIG. S3b). Furthermore, the CD thermal melting data demonstrates that the protein is very stably folded (FIG. 3). Therefore, the solubility data (FIGS. S4c & S5) combined with the crystallization screening data (FIG. 4b) suggests that MA_2137 undergoes LLPS in an essentially fully folded conformational state.

In contrast to the behavior of the WT and the D65R constructs, the 5KR and 11 KR constructs of MA_2137 showed precipitation at the highest PEG3350 concentrations used in our solubility assays, with the 11 KR construct showing stronger precipitation than the 5KR construct (FIG. 9c). These results indicate the free energy of these MA_2137 constructs is lower in the precipitated state than in the LLPS state under conditions of very low water activity, reflecting a reduction in thermodynamic solubility. However, these constructs both crystallize extremely promiscuously, with the 5KR and 11KR constructs yielding crystallization hits in ~8% and ~15% of screened conditions, respectively (FIG. 4). These results raise the possibility that the precipitate formed by the 5KR and 11 KR constructs at very high PEG3350 concentration could be in a microcrystalline state rather than an amorphously precipitated state due to the high efficacy of the Bulk KR mutations in promoting crystallization. Further research will be needed to determine whether the reduced solubility of the 5KR and 11 KR constructs reflects stabilization of crystalline states or amorphously precipitated states of MA_2137-D65R.

### Discussion

The present disclosure demonstrates the efficacy of a new method for probabilistic engineering of protein surface properties to enhance crystallization propensity based on substitution of multiple native lysine residues with arginine residues (KR substitution); multiple native asparagine residues with glutamine residues (NQ substitution); and/or multiple native aspartate residues with glutamate residues (DE substitution). [10] In this manner, a naturally occurring protein refractory to crystallization is modified with a plurality of amino acid substitutions and the resulting mutant protein displays more crystal packing interfaces and crystallizes more efficiently than the naturally occurring protein, while preserving the structure and stability of the naturally occurring protein. The amino acid substitutions may be lysine-to-arginine, aspartate-to-glutamate, and/or asparagine-to-glutamine. The rationale behind this “Bulk AA” (amino acid) substitution method is that, e.g., lysine and arginine have very similar physicochemical properties, but arginine shows substantially higher overrepresentation than lysine in a large-scale computational analysis performed of crystal structures deposited in the PDB (FIG. 1).[2]

According to certain embodiments, software and computer implemented methods employing modules and algorithms can rank lysine sites for substitution based on the redundancy-corrected count of KR substitutions observed in homologous proteins with the highest level of sequence identity (FIG. 2), based on the rationale that biological evolution selects against destabilizing and function-impairing mutations. The present disclosure demonstrates that mutations selected this way are only minimally destabilizing (FIG. 3 and Table 1) and significantly enhance crystallization propensity for two of the three tested proteins (FIG. 4). The crystals yielded by the Bulk KR method diffract strongly and enabled efficient determination of a 1.9 Å crystal structure (Table 3) for the hPDIa protein domain which does not crystallize at all in its native sequence (FIG. 4 and Table 1).

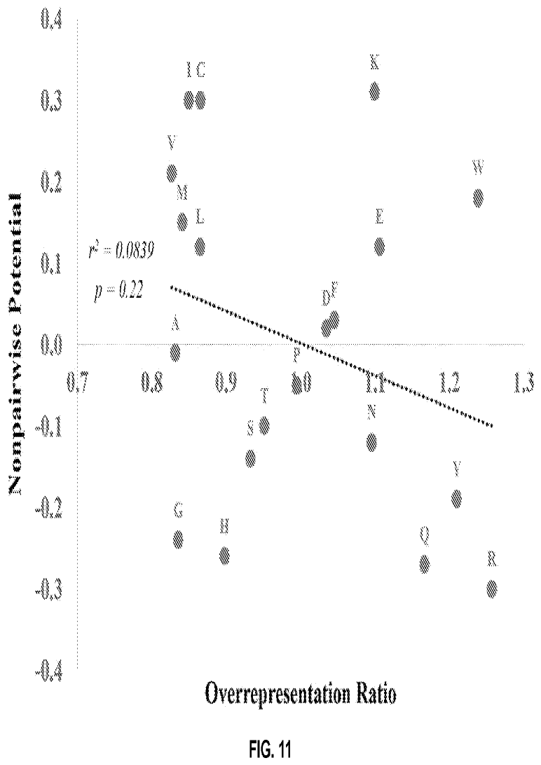

A series of previous experimental studies examined the effects of mutating surface-exposed lysine residues on crystallization propensity. These studies were guided by different conceptual premises that prioritize fundamentally different kinds of amino acid substitutions. The Surface Entropy Reduction (SER) method, which represented pioneering research on the use of rational surface mutagenesis to improve protein crystallization propensity, focused on substituting high entropy sidechains with low entropy sidechains, especially lysine-to-alanine (KA) mutations, in order to reduce the free energy penalty incurred upon immobilizing flexible surface residues in crystal-packing interfaces. The alternative approach was based on a computational analysis of the amino acids making crystal-packing interactions in a set of 233 protein crystal structures.[11] This historical analysis, which used different computational methods, produced different conclusions compared to our computational analysis of a much larger set of 87,684 crystal structures (FIG. 1). When comparing results for all 20 amino acids, there is not a statistically significant correlation between the single amino acid crystal-packing propensities deduced from the two analyses (FIG. 11). The most salient difference is that the earlier study concluded that lysine, glutamate, and tryptophan are all disfavored in crystal-packing contacts which contradicts our results reported in FIG. 1.

Two different groups subsequently tested these proposals. One group tested nine single, double, or triple KR mutations, five of which produced diffracting crystals, but only one of these five produced a crystal that diffracted to higher resolution than the wild-type protein.[12] Their overall conclusion was that KR mutations show lower efficacy in improving protein crystallization than KA mutations. The other group conducted a more extensive study that effectively led to the opposite conclusion.[13] They systematically examined substitution of 15 surface-exposed lysine residues with either glutamine or alanine, which did not yield any well-diffracting crystals. This rigorous study demonstrated that the surface mutations consistently changed the crystallization conditions that yielded hits, and weakly diffracting crystals were obtained from one KQ and one KA mutant protein, while none were obtained from the wild-type protein using the same crystallization screens. However, the KQ mutations yielded hits in those screens at the same rate at the wild-type protein withing experimental error, while the KA mutations yielded hits at a significantly reduced rate ~2/3 that of the wild-type protein. These experimental results are consistent with our computational results reported in FIG. 1, which show glutamine has a somewhat higher probability of participating in crystal-packing contacts than lysine, while alanine has a significantly lower probability of participating in crystal-packing contacts. The experimental studies summarized above highlight the probabilistic nature of the influence of single amino acid substitutions on crystallization propensity, which contributed to development of our strategy focused on engineering more consistent improvements in crystallization behavior via simultaneous introduction of multiple putative crystallization-enhancing surface mutations.

The crystal structures of Bulk KR substituted proteins show no significant conformational or stereochemical differences versus reference proteins (FIG. 8). Furthermore, the engineered arginine residues, like the native ones, make both van der Waals contacts and H-bonds in crystal-packing interfaces at substantially higher frequencies than either lysine residues or other residues (Table 2). These crystal structures were produced by the Bulk KR constructs harboring the highest number of substitutions, which were the only constructs for which any diffraction data were measured. These results support the efficacy of a streamlined pipeline for crystal structure determination in which solubility is tested for a set of constructs with an increasing number of KR mutations, but purification and crystallization screening are only performed on the construct harboring the largest number of mutations.

In summary, the biophysical results disclosed herein support bulk KR substitution being a rational and effective probabilistic strategy to engineer protein surface properties to enhance protein crystallization propensity.

The bulk KR method disclosed herein, according to some embodiments, focuses on large-scale modification of protein surface properties using mutations between amino acids that conserve qualitative physicochemical properties. The present disclosure is in some ways focused on the simultaneous introduction of multiple putative crystallization-enhancing mutations based on the hypothesis that stronger and more consistent improvements in crystallization behavior are likely to be promoted by more extensive changes in surface properties, assuming the individual changes tend to increase crystallization probability. This probabilistic surface-engineering strategy requires reliable information on the relative influence of different amino acids on crystallization propensity, and it also requires that the individual mutations do not significantly reduce protein stability so that large-scale mutagenesis does not impair protein folding and prevent effective purification. Computational analyses summarized in FIG. 1 were used to guide selection of crystallization-enhancing amino acid substitutions, leading to an initial focus on KR substitutions because of the equivalent charge and similar volume and entropy of lysine and arginine sidechains.

The present disclosure demonstrates that KR mutations at 25 sites selected based on the frequency of substitution observed in homologs shows on average a 0.54° C. reduction in apparent Tₘ per KR mutation (FIG. 3 and Table 1).

The computational results reported in FIG. 1 showed that glutamine has a somewhat higher probability of participating in crystal-packing contacts than lysine, while alanine has a significantly lower probability of participating in crystal-packing contacts. These studies further highlight the probabilistic nature of the influence of single amino acid substitutions on crystallization propensity, which contributed to development of this strategy focused on engineering more consistent improvements in crystallization behavior via simultaneous introduction of multiple putative crystallization-enhancing surface mutations.

One goal of the present large-scale surface-mutagenesis strategy is that individual mutations preserve or at most minimally perturb protein stability. This has led to the focus on physicochemically conservative substitutions and initially KR mutations versus other types of mutations. One complication with KA mutations versus KR mutations is that the grossly different physicochemical properties of alanine compared to lysine are more likely to produce significant protein destabilization. This effect impedes introduction of multiple KA mutations, while the present results (Tables 1-2) show that introduction of multiple mutations is an effective strategy to improve crystallization propensity.

The present computational analyses raise additional questions about the efficacy of using KA mutations. Alanine is significantly underrepresented in crystal-packing interfaces, while lysine is significantly overrepresented (FIG. 1). These observations are consistent with the results of computational analyses demonstrating that, for all amino acids, increasing solvent exposure correlates strongly with increasing probability of making a crystal-packing interaction. The low solvent exposure of alanine residues at most sites in proteins is therefore consistent with the strong underrepresentation of alanine in crystal-packing interfaces (FIG. 1). Additional computational analyses show that some alanine-containing sequences located in α-helix-capping motifs and short surface loops are significantly overrepresented in crystal-packing interfaces. These results suggest that the influence of KA mutations on crystallization propensity is likely to depend strongly on local structural context and may involve more complex physicochemical effects than surface entropy reduction.

The conceptual foundation of the crystallization engineering method presently disclosed is fundamentally unique. As indicated above, it focuses on probabilistic reengineering of protein surface properties via simultaneous substitution of multiple amino acids with similar physicochemical properties but different propensity to make crystal-packing interactions based on large-scale computational analyses of previously determined crystal structures (FIG. 1).[14] Significant improvement in the crystallization properties of two out of three target proteins was demonstrated when the method was applied using a streamlined experimental design in which a single mutant construct of each target was purified and subjected to crystallization screening (FIGS. 4-5, Tables 1-2, and Table 3). For each target, the expression and solubility properties were evaluated for a series of constructs containing an increasing number of physiochemically conservative crystallization-enhancing mutations, which is readily done in parallel for at least a dozen constructs, but exclusively the soluble construct with greatest number of crystallization-enhancing mutations was purified, which involves substantially more labor-intensive methods. This streamlined experimental protocol led to solution of a high-resolution crystal structure (FIG. 5b and Table 3) for the hPDIa domain, a human drug target that does not yield any crystal hits with its native sequence (FIG. 4b).

Given the success of the Bulk KR method in improving protein crystallization behavior, the present computational analysis of crystal-packing interactions in the PDB (FIG. 1) suggests several related strategies with promise to improve crystallization behavior based on the same conceptual approach. Aspartate and glutamate frequently substitute for one another in the course of evolution due to their very similar physicochemical properties, but glutamate shows over 2-fold higher overrepresentation in crystal-packing interfaces (FIG. 1). A similar trend relative to crystal packing interactions is observed for asparagine and glutamine, which also have very similar physicochemical properties. These observations suggest bulk aspartate-to-glutamate (DE) and asparagine-to-glutamine (NQ) substitutions are also likely to improve crystallization propensity.

In the case of these substitutions, the higher entropy of the sidechain with greater crystal-packing propensity will tend to thermodynamically oppose immobilization in a crystal-packing interface. This factor does not apply to bulk lysine-to-arginine substitution due to the very similar entropy of these sidechains. However, the inventor's computational analyses of crystal-packing interactions in the PDB shows that high-entropy sidechains mediating crystal-packing interactions tend to participate in salt-bridging and H-bonding interactions with nearby residues in the primary sequence, especially at ±3 and ±4 positions in α-helices and ±2 positions in β-strands. These interactions likely reduce the entropy of the sidechains in the isolated protein molecules, which will reduce or eliminate entropy loss due to immobilization in a crystal-packing interface. Therefore, bulk DE and NQ substitution seems likely to be most effective when residues with potential salt-bridging or H-bonding partners at ±3 and ±4 positions in α-helices or ±2 positions in β-strands are prioritized for substitution.

### Materials and Methods

### Site-Selection Software and Input Sequence Alignment Format

According to certain embodiments, the present disclosure relates to a Bulk Amino Acid Substitution Crystallization Propensity Application comprising an Amino Acid Mutation Site-Selection module and GUI module. For example, the disclosure includes a computer-implemented method performed via software or program application or modules such as, for example, a python program that automatically generates a suggested order for mutation of lysine to arginine. In one embodiment, a Python application that automatically generates a suggested order for mutation of lysine residues to arginine incorporates routines from the BioPython package.[15] The BioPython tools for molecular biology can be accessed at https://biopython.org/which is hereby incorporated by reference. The Bulk Amino Acid Substitution Crystallization Propensity Application, Amino Acid Mutation Site-Selection module, and GUI module program code is available for download from Github archive (https://github.com/huntmolecularbiophysicslab/pxengineering), and it can also be run interactively using the protein crystallization engineering webserver (http://www.pxengineering.org). The sites, program code, and information and data represented by said sites and program code are hereby incorporated by reference as if fully restated in their entireties herein. The systems, application, and modules disclosed herein can be coded or designed differently or functionalities can be combined with other systems, applications, and/or modules to perform the claimed methods and computer implemented methods as would be understood by a skilled artisan.

According to some embodiments, the application can accept input of an alignment of homologous sequences, for example, in Clustal format. The first line of files in this ASCII format start with the words “CLUSTAL W” or “CLUSTALW”. Blank lines then separate sequential blocks of text containing aligned sequence segments with one sequence per line. Each of those lines starts with a string containing the sequence name, which is followed by blank spaces and then up to 60 single-letter amino acid codes. Each line can contain additional blank spaces followed by a residue number. Each block optionally ends with a line containing characters encoding the degree of sequence conservation at each position in the alignment.

The Clustal program generates a multiple sequence alignment (MSA) in a specific file format. This MSA serves as a visual representation of the similarity between input protein sequences and aids in identifying potentially functionally or structurally significant regions.

The core of the Clustal output is the alignment itself. Generally, each sequence occupies a distinct row, with identical or similar residues positioned in corresponding columns. Gaps, represented by dashes (-), are strategically inserted to maximize the alignment of these similar residues. Additionally, each sequence is labeled with its corresponding name and total length for easy identification.

Clustal also incorporates conservation scores, often displayed as asterisks (*) or other symbols, to indicate the level of conservation at each position within the alignment. Positions with high conservation scores suggest that the residue at that location is highly conserved across the aligned sequences, potentially signifying functional or structural importance.

Beyond the core alignment data, the Clustal output format includes additional details. The alignment is typically saved as a text file with a specific extension, such as “.aln” (CLUSTAL format), depending on the user-chosen output format (other options include FASTA, PHYLIP etc.). To enhance readability, the alignment might be further divided into sections called “blocks.” Each block displays a portion of the aligned sequences, with the total number of residues shown at the end of the block. Information lines may also be present, summarizing the alignment process or offering additional details like the alignment method used or user-defined parameters.

### GPU Acceleration of Sequence Identity Calculation

According to certain embodiments, the software implemented method can be performed by an application, for example, an application programmed in Python, to accelerate calculation of the absolute percent identity between two sequences by parallelizing site-by-site comparison or sequence comparison on a Graphical Processing Unit (GPU) chip using a custom reduction kernel written using the CuPy library. In brief, each pair of aligned amino acids in two sequences can be sent to a separate GPU core. If the 1-character amino acid codes in both sequences match each other and neither is empty due to a gap in the sequence alignment, that core stores a value of 1 in its register, which is otherwise set to zero. The values in the registers for all sites are then summed using parallel reduction to count the number of identical amino acids in the sequence. Details can be found in the code at https://github.com/huntmolecularbiophysicslab/pxengineering. The code, data, and information represented by this link is hereby incorporated by reference in its entirety as if fully restated herein.

### Prioritization of Mutation Sites Based on Redundancy-Corrected Counts of KR Mutations Observed in Homologous Proteins

To compensate for outright redundancy as well as inhomogeneous phylogenetic sampling in sequence databases, the application and computer implemented method can include, for example, two redundancy-compensation calculations on the set of sequences in each percent-identity bin having an arginine substitution at a specific lysine site in the target protein. Both calculations can use the same heuristic estimate for the probability of evolutionary resampling at an aligned site between two sequences i and j with an overall fraction of f_{id} identical residues at all aligned sites:

$${{P}_{site - resampling} ( {f}_{i d} )}_{i j} = \{ \begin{matrix}1. for {f}_{id} \leq {f}_{min} \\ ( \frac{1 - {f}_{id}}{1 - {f}_{min}} ) for {f}_{id} > {f}_{min}\end{matrix} \} .$$

P_{site-resampling} is essentially the probability that a mutation has occurred in the codon for a single amino acid in one protein sequence compared to another. The mutation can be silent, so the sequence does not necessarily change, but this effectively measures the probability of independent mutations occurring at a single site between two homologous proteins.

Generally assuming fₘᵢₙ=0.3, the following calculation can give a redundancy-reduced estimate C_{R} of arginine counts using the following formula in which the summation is performed over all unique pairs of the N sequences in the bin having an arginine substitution at one lysine site in the target protein:

$${C}_{R} = ( \frac{2}{N} {\sum }_{i < j} {{P}_{site - resampling} ( {f}_{i d} )}_{i j} ) + 1.$$

This calculation is extremely rapid and includes all homologs but is only rigorously accurate in the limiting cases of full redundancy or full independence. A different calculation can give a rigorous estimate of the expectation value for the number of independent observations of arginine based on combinations of the heuristic probabilities of being resampled or not being resampled for all unique pairs among the seven most diverged sequences in each bin that have arginine at a specific lysine site. These sequences can be identified by taking the single sequence with the lowest percent identity to the target sequence and then progressively adding the sequence with the lowest average percent identity to those already selected. The details of the implementation can be found in the code at https://github.com/huntmolecularbiophysicslab/pxengineering.

### Protein Expression and Purification

Following identification on lysine sites for lysine to arginine substitution in a naturally occurring protein sequence according the above methods, protein coding nucleic acid sequences harboring Bulk KR Substitutions were synthesized with a short C-terminal hexahistidine tag (LEHHHHHH), cloned under the control of the T7 RNA polymerase promoter in the pET21_NESG expression plasmid, and then transformed into BL21(DE3) Rosetta E. coli cells. Protein expression was induced with 1 mM IPTG for 4 hours at 30° C. in Terrific Broth.[16] Cells were pelleted by centrifugation at 4,000 rpm for 25 min at 4° C. and resuspended on ice in 10 mM imidazole, 300 mM NaCl, 1 mM TCEP, 5% (w/v) glycerol, 50 mM NaH₂PO₄, pH 7.5, prior to cell lysis by probe sonication. The supernatant following 15,000 rpm centrifugation at 4° C. was mixed with Ni-NTA resin and incubated at 4° C. for 1 hour. The mixture was then transferred into a column and washed with the same buffer containing a higher 100 mM imidazole concentration prior to elution of the protein in 6 ml of the same buffer containing 250 mM imidazole. A 1 mL aliquot of eluted protein was concentrated to 500 μl using an Amicon 10 kDa centrifugal filter and loaded via a 1 ml loop for onto a Superdex 200 Increase 10/300 GL gel filtration column equilibrated in 100 mM NaCl, 10 mM DTT, 10 mM Tris-CI, pH 7.5. Protein-containing fractions were concentrated to ~15 mg/ml based on a priori sequence-based extinction coefficients and OD280 nm values measured using a Nanodrop spectrophotometer (ThermoFisher), and the concentrated protein was immediately flash-frozen in aliquots in liquid nitrogen prior to storage at −80° C. pending use.

### Thermal Stability Assays Using CD Spectroscopy

An Applied Photophysics Chirascan V100 spectropolarimeter with a Peltier-jacketed cell holder was used to collect circular dichroism (CD) spectra spanning 200-250 nm serially during a 3° C./min thermal ramp nominally running from 10-84° C. Data were measured from protein samples in a 0.5 mm quartz cuvette in 1 nm increments using a 0.25 integration time per point and a 1 nm bandwidth, corresponding to 35 sec per spectrum. Measurements of the actual cell temperature during the experiment, which were used for data display and analysis, indicated the actual range of the temperature ramp was from ~11-78° C. for all samples. Protein samples were diluted to 2 mg/ml using gel filtration buffer lacking DTT (i.e., 100 mM NaCl, 10 mM Tris-CI, pH 7.5) with the addition of 1 mM SAH for the MA_237 constructs. Global curve fitting of spectral data during the thermal ramp was performed from 215-230 nm using the program GLOBAL3 using double linear baseline correction (i.e., before and after the observed transitions) to extract the thermodynamic parameters and melting temperatures (Table 1 and FIG. 3). Each dataset was analyzed using the smallest number of transitions showing approximately random directions for the residuals for adjacent points in the CD vs. measured temperature plane. Because the reversibility of the unfolding transitions was not evaluated, the inferred Tₘ and ΔH_{vH} (DH_{vH}) values listed in Table 1 cited in the text are labeled as apparent.

### Solubility Assays

Protein stock solutions were diluted to working concentration in the same buffer used for gel-filtration chromatography containing different weight/volume concentrations of PEG3350. Following a 60-minute incubation at room temperature, the samples were spun for 10 min at 14,000 RPM in a microfuge to pellet particulates, and the concentration of protein in the supernatant was measured using the optical density at 280 nm measured in a Nanodrop spectrophotometer based on the a priori extinction coefficient. The centrifugation and measurement of concentration in the supernatant were repeated 24 hours later to ensure equilibrium had been reached.

### Protein Crystallization Screening

For each target protein, the parental construct and the construct containing the largest number of KR mutations that could be purified, but not the constructs containing fewer KR mutations, were submitted for crystallization screening using the standard protocol at the High-Throughput Crystallization Screening Center at the Hauptman-Woodward Medical Research Institute (https://hwi.buffalo.edu/high-throughput-crystallization-center/). Protein stocks were adjusted to 15 mg/ml concentration prior to mixing 1:1 with the well solution for microbatch under-oil crystallization screening. Crystallization reactions were imaged in a Rock Imager before protein addition and at one day, one week, two weeks, three weeks, four weeks, and six weeks after reaction setup using parallel brightfield microscopy, ultraviolet two-photon-exited fluorescence microscopy, and SONICC (second-order harmonic imaging of chiral crystals) microscopy. Hits were identified based on visual observation of refractive supramolecular aggregates in brightfield microscopy images that had corresponding features in either fluorescence or SONICC microscopy images or both. Hits were characterized as “High Quality” based on the judgement of an expert protein crystallographer that it would likely be straightforward to reproduce the crystals and optimize them to sufficient size to mount for measurement of x-ray diffraction data.

Optimally, crystals are obtained by this method that can yield 3.0 Å resolution or better in x-ray diffractometry are obtained. In an embodiment, crystals are obtained by this method with 2.5 Å resolution or better, or 1.9 Å resolution or better.

### Protein Crystal Optimization

A small subset of the crystal hits for hPDIa-9KR and MA_2137-D65R-11 KR were reproduced using the microbatch-under-oil method at 4° C. and 18° C., and they were subsequently optimized by seeding. The crystal used for determination of the hPDIa-9KR structure was grown by mixing the 15 mg/ml stock solution at a 2:1 volume ratio with a crystallization reagent comprising 24% (w/v) PEG 20k, 0.1 M potassium thiocyanate, 0.1 M MES, pH 6. The crystal used for determination of the MA_2137-D65R-11 KR structure was grown by mixing the 15 mg/ml stock solution at a 1:1 volume ratio with a crystallization reagent comprising 30% (w/v) PEG 1k, 0.1 M HEPES, pH 7.5. All crystals were transferred into a similar crystallization solution supplemented with 20% (v/v) ethylene glycol prior to mounting and flash-freezing in liquid nitrogen.

### Crystal Structure Determination and Refinement

X-ray diffraction data were collected from single crystals of PDIa-9M and MA_2137-D65R-11 using, respectively, the NE-CAT 24-ID-E and 24-ID-C beam lines at the Advanced Photon Source (Table 3). The images were processed and scaled using XDS.[17] The structure of hPDIa-9KR was solved by molecular replacement using the program MOLREP 132 employing a search model comprising the first domain in the crystal structure of full-length hPDI (PDB id 4EKZ). The structure of MA_2137-D65R-11 KR was solved using the same methods employing the structure of MA_2137-D65R (PDB id 6MRO) as the search model. Both structures were refined (Table 3) using PHENIX 4[18] in conjunction with manual rebuilding in XtalView 133[19] and COOT.[20]

### REFERENCES

(1) Jumper, J.; Evans, R.; Pritzel, A.; Green, T.; Figurnov, M.; Ronneberger, O.; Tunyasuvunakool, K.; Bates, R.; \hat{Z}idek, A.; Potapenko, A.; Bridgland, A.; Meyer, C.; Kohl, S. A. A.; Ballard, A. J.; Cowie, A.; Romera-Paredes, B.; Nikolov, S.; Jain, R.; Adler, J.; Back, T.; Petersen, S.; Reiman, D.; Clancy, E.; Zielinski, M.; Steinegger, M.; Pacholska, M.; Berghammer, T.; Bodenstein, S.; Silver, D.; Vinyals, O.; Senior, A. W.; Kavukcuoglu, K.; Kohli, P.; Hassabis, D. Highly Accurate Protein Structure Prediction with AlphaFold. Nature 2021, 596 (7873), 583-589. https://doi.org/10.1038/s41586-021-03819-2.
(2) Naumov, V.; Price, W. N.; Handelman, S. K.; Hunt, J. F. Engineering Surface Epitopes to Improve Protein Crystallization. U.S. Pat. No. 10,294,266, May 21, 2019.
(3) Banayan, N. E.; Loughlin, B. J.; Singh, S.; Forouhar, F.; Lu, G.; Wong, K.-H.; Neky, M.; Hunt, H. S.; Bateman Jr, L. B.; Tamez, A.; Handelman, S. K.; Price, W. N.; Hunt, J. F. Systematic Enhancement of Protein Crystallization Efficiency by Bulk Lysine-to-Arginine (KR) Substitution. Protein Science 2024, 33 (3), e4898. https://doi.org/https://doi.org/10.1002/pro.4898.
(4) Khan, M. M. G.; Simizu, S.; Kawatani, M.; Osada, H. The Potential of Protein Disulfide Isomerase as a Therapeutic Drug Target. Oncology Research Featuring Preclinical and Clinical Cancer Therapeutics 2011, 19 (10), 445-453. https://doi.org/10.3727/096504011Xl3123323849717.
(5) Hoffstrom, B. G.; Kaplan, A.; Letso, R.; Schmid, R. S.; Turmel, G. J.; Lo, D. C.; Stockwell, B. R. Inhibitors of Protein Disulfide Isomerase Suppress Apoptosis Induced by Misfolded Proteins. Nat Chem Biol 2010, 6 (12), 900-906. https://doi.org/10.1038/nchembio.467.
(6) Goedken, E. R.; Marqusee, S. Co-Crystal of Escherichia Coli RNase HI with Mn2+ Ions Reveals Two Divalent Metals Bound in the Active Site. Journal of Biological Chemistry 2001, 276 (10), 7266-7271. https://doi.org/i0.1074/jbc.M009626200.
(7) Price II, W. N.; Chen, Y.; Handelman, S. K.; Neely, H.; Manor, P.; Karlin, R.; Nair, R.; Liu, J.; Baran, M.; Everett, J.; Tong, S. N.; Forouhar, F.; Swaminathan, S. S.; Acton, T.; Xiao, R.; Luft, J. R.; Lauricella, A.; DeTitta, G. T.; Rost, B.; Montelione, G. T.; Hunt, J. F. Understanding the Physical Properties That Control Protein Crystallization by Analysis of Large-Scale Experimental Data. Nat Biotechnol 2009, 27 (1), 51-57. https://doi.org/10.1038/nbt.1514.
(8) Budziszewski, G. R.; Snell, M. E.; Wright, T. R.; Lynch, M. L.; Bowman, S. E. J. High-Throughput Screening to Obtain Crystal Hits for Protein Crystallography. Journal of Visualized Experiments 2023, No. 193. https://doi.org/10.3791/65211.
(9) Cruickshank, D. W. J. The Required Precision of Intensity Measurements for Single-Crystal Analysis. Acta Crystallogr 1960, 13 (10), 774-777. https://doi.org/10.1107/S0365110X60001874.
(10) Lowe, D. Getting Proteins to Crystallize. Science (1979) 2023.
(11) Dasgupta, S.; lyer, G. H.; Bryant, S. H.; Lawrence, C. E.; Bell, J. A. Extent and Nature of Contacts between Protein Molecules in Crystal Lattices and between Subunits of Protein Oligomers. Proteins: Structure, Function, and Genetics 1997, 28 (4), 494-514. https://doi.org/10.1002/(SICI)1097-0134(199708)28:4<494::AID-PROT4>3.0.CO; 2-A.
(12) Czepas, J.; Devedjiev, Y.; Krowarsch, D.; Derewenda, U.; Otlewski, J.; Derewenda, Z. S. The Impact of Lys→Arg Surface Mutations on the Crystallization of the Globular Domain of RhoGDI. Acta CrystallogrD Biol Crystallogr 2004, 60 (2), 275-280. https://doi.org/10.1107/SO907444903026271.
(13) Anstrom, D. M.; Colip, L.; Moshofsky, B.; Hatcher, E.; Remington, S. J. Systematic Replacement of Lysine with Glutamine and Alanine in Escherichia Coli Malate Synthase G: Effect on Crystallization. Acta Crystallogr Sect F Struct Biol Cryst Commun 2005, 61 (12), 1069-1074. https://doi.org/i0.1107/S1744309105036559.
(14) Banayan, N. E.; Loughlin, B. J.; Singh, S.; Forouhar, F.; Lu, G.; Wong, K.-H.; Neky, M.; Hunt, H. S.; Bateman Jr, L. B.; Tamez, A.; Handelman, S. K.; Nicholson Price, W.; Hunt, J. F. Systematic Enhancement of Protein Crystallization Efficiency by Bulk Lysine-to-Arginine (KR) Substitution Bulk K-to-R Substitution Enhances Crystallization. BioRxiv. 2023. https://doi.org/10.1101/2023.06.03.543563.
(15) Cock, P. J. A.; Antao, T.; Chang, J. T.; Chapman, B. A.; Cox, C. J.; Dalke, A.; Friedberg, I.; Hamelryck, T.; Kauff, F.; Wilczynski, B.; de Hoon, M. J. L. Biopython: Freely Available Python Tools for Computational Molecular Biology and Bioinformatics. Bioinformatics 2009, 25 (11), 1422-1423. https://doi.org/10.1093/bioinformatics/btpl63.
(16) Terrific Broth. Cold Spring Harb Protoc 2015, 2015 (7), pdb.rec087874. https://doi.org/10.1101/pdb.rec087874.
(17) Kabsch, W. Integration, Scaling, Space-Group Assignment and Post-Refinement. Acta Crystallogr D Biol Crystallogr 2010, 66 (2), 133-144. https://doi.org/10.1107/S0907444909047374.
(18) Liebschner, D.; Afonine, P. V.; Baker, M. L.; Bunkóczi, G.; Chen, V. B.; Croll, T. I.; Hintze, B.; Hung, L.-W.; Jain, S.; McCoy, A. J.; Moriarty, N. W.; Oeffner, R. D.; Poon, B. K.; Prisant, M. G.; Read, R. J.; Richardson, J. S.; Richardson, D. C.; Sammito, M. D.; Sobolev, O. V.; Stockwell, D. H.; Terwilliger, T. C.; Urzhumtsev, A. G.; Videau, L. L.; Williams, C. J.; Adams, P. D. Macromolecular Structure Determination Using X-Rays, Neutrons and Electrons: Recent Developments in Phenix. Acta Crystallogr D Struct Biol 2019, 75 (10), 861-877. https://doi.org/10.1107/S2059798319011471.
(19) McRee, D. E. XtalView/Xfit—A Versatile Program for Manipulating Atomic Coordinates and Electron Density. J Struct Biol 1999, 125 (2-3), 156-165. https://doi.org/10.1006/jsbi.1999.4094.
(20) Casañal, A.; Lohkamp, B.; Emsley, P. Current Developments in Cootfor Macromolecular Model Building of Electron Cryo-microscopy and Crystallographic Data. Protein Science 2020, 29 (4), 1055-1064. https://doi.org/10.1002/pro.3791.

1. A method to improve the number and quality of obtainable protein crystal forms comprising mutationally substituting, on a naturally occurring protein refractive to crystallization:
a. multiple native lysine residues with arginine residues;
b. multiple native asparagine residues with glutamine residues; and/or
c. multiple native aspartate residues with glutamate residues.

2. The method of claim 1, in which sites for substitution are selected based on the frequencies of the same substitutions being observed in naturally evolved proteins, with more frequently substituted sites being selected before less substituted sites.

3. The method of claim 1, in which sites for substitution are selected based on the redundancy-corrected frequencies of the same substitutions being observed in naturally evolved proteins, with more frequently substituted sites being selected before less substituted sites.

4. The method of claim 3, in which non-redundancy is calculated based on the probability of evolutionary resampling at individual amino acid sites between multiple homologs bearing the same substitution based on their overall percent identity relative to the percentage of sites that cannot be substituted due to loss of structure or function, wherein the overall percent identity is about 30%.

5. The method of claim 1, in which sites for substitution are prioritized based on frequencies calculated in successive bins comprising non-overlapping ranges of decreasing percent sequence identity between homologs and the target protein.

6. The method of claim 1, in which sites with complementary hydrogen-bonding groups at ±2 residues in predicted or observed beta-strand structures or at ±3 or ±4 residues in predicted or observed alpha-helical structures are prioritized ahead of sites without such potential hydrogen-bonding partners even if the observed substitution frequencies are lower, but maintaining the substitution-frequency-based priorities within each group.

7. The method of claim 1, in which a set of mutant proteins harboring increasing numbers of substitutions with progressively decreasing priority from sequence-based ranking are expressed and evaluated for soluble protein expression.

8. The method of claim 7, in which the soluble protein harboring the greatest number of substitutions is purified and subjected to crystallization screening.

9. The method of claim 8, in which the other soluble proteins harboring multiple lysine-to-arginine, aspartate-to-glutamate, or asparagine-to-glutamine substitutions or combinations thereof are purified and subjected to crystallization screening.

10. The method of claim 1, wherein a mutant protein sequence is obtained, and the sequence is synthesized, expressed in an expression system, purified, crystallized, and an x-ray crystal structure is obtained using x-ray diffraction methods.
